(19) United States
(12) Patent Application Publication
(10) Pub. No.: US 2016/0304618 A1
(43) Pub. Date: Oct. 20, 2016

(54) ANTIBODIES FOR DIAGNOSIS OF ACUTE MYELOID LEUKEMIA

(71) Applicant: FRAUNHOFER-GESELLSCHAFT ZUR FÖRDERUNG DER ANGEWANDTEN FORSCHUNG E.V., Munich (DE)

(72) Inventors: Stefan Barth, Aachen (DE); Mehmet Kemal Tur, Gießen (DE); Jenny Fitting, Aachen (DE)

(21) Appl. No.: 14/913,843

(22) PCT Filed: Aug. 27, 2014
(86) PCT No.: PCT/EP2014/068116

§ 371 Date: Feb. 23, 2016

(30) Foreign Application Priority Data
Aug. 27, 2013 (EP) 13181874.2

(51) Int. Cl. C07K 16/30; G01N 33/574

(52) U.S. Cl. CPC C07K 16/30; G01N 33/57426; C07K 2317/565; C07K 2317/622; C07K 2317/33; C07K 2317/92

(57) ABSTRACT

A polypeptide in particular an antibody or antibody fragment is disclosed wherein the polypeptide is corresponding to certain complementarity determining regions CDR1, CDR2 and CDR3 of a heavy chain V_{H} and a light chain V_{L} of an antibody as well as a compound comprising the polypeptide, its use as diagnostic agent for acute myeloid leukemia subtype M2 and a kit comprising the compound.

The present invention pertains to a polypeptide in particular an antibody or antibody fragment wherein the polypeptide is corresponding to certain complementarity determining regions CDR1, CDR2 and CDR3 of a heavy chain V_{H} and a light chain V_{L} of an antibody as well as a compound comprising the polypeptide, its use as diagnostic agent and a kit comprising the compound of the invention.

Adult acute myeloid leukemia (AML) is a highly heterogeneous stem cell malignancy characterised by the clonal expansion of immature myeloid precursor cells. AML may emerge de novo, following other haematopoietic malignancies or after the cytotoxic therapy of other disorders. Although the cancer treatment regime has been improved significantly over the last decades, the 5-year survival rate still ranges from 24% to 70% and strongly depends on the diagnosed subtype. The identification of AML subtype signatures is the first important step in AML treatment because the outlook for a particular patient depends on whether he or she has the subtype that is favourable, intermediate or unfavourable.

Since fast and precise diagnosis is of high importance, an object of the present invention is the provision of tools for diagnosis of AML subtype M2 specific diagnoses.

WO 2005/111623 A1 discloses a marker for AML, binding molecules that specifically bind to the new marker, nucleic acid molecules encoding the binding molecules and compositions comprising the binding molecules. The binding molecules capable of specifically binding to the marker can be used in the diagnosis of AML. This reference is silent with respect to the disclosure of an antibody specific to subtype M2.

US 2008/0095780 A1 discloses tumor-associated antigens, binding molecules that specifically bind to the antigens, nucleic acid molecules encoding the binding molecules, compositions comprising the binding molecules and methods of identifying or producing the binding molecules. The tumor-associated antigen are expressed on cancer cells and binding molecules capable of specifically binding to the antigens can be used in the diagnosis, prevention and/or treatment of cancer. No antibody is disclosed which is specific for AML subtype 2.

WO 2011/036183 A2 discloses antibodies to the tumor-associated antigen CD33 and to the use thereof for immunotargeting CD33-positive cells. The antibodies are suitable for use in the field of medicine, pharmaceuticals, and biomedical research. The antibodies are characterized by a high affinity for human CD33, of the order of magnitude of 10-10 mol/l. The CDR sequences are suitable in particular for producing recombinant fragments (such as scFv fragments or bispecific antibodies) and for immunotargeting, due to the high affinity thereof. Further disclosed is the use of the antibody for producing a medication for therapeutic and/or diagnostic application for illnesses associated with the expression of CD33, particularly for acute myeloid leukemia (AML). No subtype M2 specificity is addressed or disclosed.

US 2005/069955 A1 discloses antibodies or fragments thereof that bind to cancer cells and is important in physiological phenomena, such as cell rolling and metastasis. Therapeutic and diagnostic methods and compositions using such antibody fragments thereof are also disclosed. The methods and compositions according to the present invention can be used in targeting therapeutic agents and in diagnosis, prognosis, and staging of and therapy for such diseases as cancer, including tumor growth and metastasis, leukemia, auto-immune disease, and inflammatory disease. Also provided is a library of immunoglobulin binding domains having a diverse antigen-binding domain for complementary binding, wherein the library has diversity only in heavy chain CDR3. In regard to leukemia no specific antibody for AML subtype M2 is disclosed.

The object underlying the present invention is accomplished by a polypeptide comprising an antibody or antibody fragment wherein the polypeptide is corresponding to complementarity determining regions CDR1, CDR2 and CDR3 of a heavy chain V_{H} and a light chain V_{L} of an antibody, the complementarity determining regions comprising
the CDR 1 region of the heavy chain V_{H} is defined by a sequence of 5 amino acids wherein the amino acids have side chain polarities and charges at a pH of 7.4, and the amino acids of the amino acid sequence are symbolized by a symbol as represented by the formula

PO_{N}/PO_{N}/NP_{N}/NP_{N}/PO_{N}

and the amino acids are linked via peptide bonds,
the CDR 2 region of the heavy chain V_{H} is defined by a sequence of 17 amino acids wherein the amino acids have side chain polarities and charges at a pH of 7.4, and the amino acids of the amino acid sequence are symbolized by a symbol as represented by the formula

PO_{N}/NP_{N}/PO_{N}/PO_{N}/BP⁺ or NP_{N}/PO_{N}/BP⁺ or PO_{N}/BP⁺/PO_{N}/NP_{N}/PO_{N}/NP_{N}/AP⁻/PO_{N}/NP_{N}/BP⁺/PO_{N}

and the amino acids are linked via peptide bonds,
the CDR 3 region of the heavy chain V_{H} is defined by a sequence of 7 amino acids wherein the amino acids have side chain polarities and charges at a pH of 7.4, and the amino acids of the amino acid sequence are symbolized by a symbol as represented by the formula

NP_{N}/NP_{N} or BP⁺/BP⁺ or NP_{N}/BP^{+ or PO}_{N}/NP_{N}/AP⁻/PO_{N}

and the amino acids are linked via peptide bonds,
the CDR 1 region of the light chain V_{L} is defined by a sequence of 11 amino acids wherein the amino acids have side chain polarities and charges at a pH of 7.4, and the amino acids of the amino acid sequence are symbolized by a symbol as represented by the formula

BP⁺/NP_{N}/PO_{N}/PO_{N}/PO_{N}/NP_{N}/PO_{N}/PO_{N}/PO_{N}/NP_{N}/PO_{N}

and the amino acids are linked via peptide bonds,
the CDR 2 region of the light chain V_{L} is defined by a sequence of 7 amino acids wherein the amino acids have side chain polarities and charges at a pH of 7.4, and the amino acids of the amino acid sequence are symbolized by a symbol as represented by the formula

NP_{N} or BP⁺/NP_{N}/PO_{N}/BP⁰ or NP_{N}/NP_{N}/PO_{N}/PO_{N}

and the amino acids are linked via peptide bonds,
the CDR 3 region of the light chain V_{L} is defined by a sequence of 9 amino acids wherein the amino acids have side chain polarities and charges at a pH of 7.4, and the amino acids of the amino acid sequence are symbolized by a symbol as represented by the formula

PO_{N}/PO_{N}/NP_{N} or BP⁺/BP⁺ or NP_{N}/PO_{N} or BP⁺/PO_{N}/NP_{N}/NP_{N}/PO_{N}

and the amino acids are linked via peptide bonds,
wherein the amino acids of the formulas are proteinogenic amino acids and the symbols have the meaning:
PO_{N} represents an amino acid having a polar side chain polarity and a neutral side chain charge at pH 7.4;
NP_{N} represents an amino acid having a non-polar side chain polarity and a neutral side chain charge at pH 7.4;
BP⁺ represents an amino acid having a basic polar side chain polarity and a positive side chain charge at pH 7.4;
BP⁰ represents an amino acid having a basic polar side chain polarity and a predominantly neutral side chain charge at pH 7.4; and
AP⁻ represents an amino acid having an acidic polar side chain polarity and a negative side chain charge at pH 7.4.

In an embodiment of the present invention the polypeptide of the invention
PO_{N} represents an amino acid selected from the group consisting of asparagine, glutamine, serine, threonine, and tyrosine;
NP_{N} represents an amino acid selected from the group consisting of alanine, cysteine, glycine, isoleucine, leucine, methionine, phenylalanine, proline, tryptophane, and valine;
BP⁺ represents arginine or lysine;
BP⁰ represents histidine; and
AP⁻ represents aspartic acid or glutamic acid.

In a further embodiment the polypeptide of the present invention comprises an antibody or antibody fragment comprising in its
heavy chain CDR 1 a peptide having at least 80% homology to the peptide of the amino acid sequence of SEQ ID NO 1;
heavy chain CDR 2 a peptide having at least 85% homology to the peptides of the amino acid sequences SEQ ID NO 2 or SEQ ID NO 3;
heavy chain CDR 3 a peptide having at least 85% homology to the peptides of the amino acid sequences SEQ ID NO 4 or SEQ ID NO 5.

In yet another embodiment the polypeptide of the present invention comprises an antibody or antibody fragment comprises in its
light chain CDR 1 a peptide having at least 80% homology to the peptide of the amino acid sequence of SEQ ID NO 6;
the light chain CDR 2 a peptide having at least 70% homology to the peptides of the amino acid sequences of SEQ ID NO 7 or SEQ ID NO 8;
a peptide having at least 50% homology to the peptides of the amino acid sequences of SEQ ID NO 9 or SEQ ID NO 10.

Typically, in the polypeptide of the invention the amino acid sequence of the heavy chain CDR 1 is the sequence of SEQ ID NO 1, the amino acid sequence of the heavy chain CDR 2 is the sequence of SEQ ID NO 2 or SEQ ID NO 3, and the amino acid sequence of the heavy chain CDR 3 is the sequence of SEQ ID NO 4 or SEQ ID NO 5 and/or the amino acid sequence of the light chain CDR 1 is the sequence of SEQ ID NO 6, the amino acid sequence of the light chain CDR 2 is the sequence of SEQ ID NO 7 or SEQ ID NO 8, and the amino acid sequence of the light chain CDR 3 is the sequence of SEQ ID NO 9 or SEQ ID NO 10.

In a particular embodiment of the invention in the polypeptide the CDR1, CDR2 and CDR3 of the heavy chain of the variable region of an antibody v_{H} and CDR1, CDR2 and CDR3 of the light chain of the variable region of an antibody v_{L} are linked with each other via a linker structure. Typically, according to the invention the linker structure is (Gly₄Ser)₃.

In a further particular embodiment the polypeptide is an antibody or a recombinant antibody, in particular a single-chain variable fragment (scFv).

Subject matter of the present invention is also a compound comprising the polypeptide of the invention comprising a detectable label.

In a particular embodiment of the compound of the invention the detectable label is selected from the group consisting of fluorescent dyes, such as fluorescein, rhodamine, coumarine, and cyanine and derivatives thereof; gamma rays emitting radioisotopes, in particular iodine-131, lutetium-177, yttrium 90; a quantum dot composed of heavy metals, in particular CdSe or InGaP; noble metal nanoclusters composed of least three, in particular 8-12 gold or silver atoms, or synthetic fluorophores captured in nanoparticles made from silicon dioxide; super paramagnetic iron oxid particles for MRI based molecular imaging; fluorescent proteins like GFP or dsRED or derivatives thereof; enzymes, such as alkaline phosphatase, peroxidases and galactosidases.

In yet another embodiment of the compound of the invention the polypeptide of the invention is linked with the detectable label by means of a chemical linking group.

For the coupling the detectable label and the polypeptide domain a chemical linking group can be arranged between the detectable label and polypeptide of the invention. The linking of the detectable label can be performed by conjugation of the respective moieties with the peptide of the invention. It is also possible to use the technology as provided by the disclosure of WO2009/013359 incorporated by reference.

The great potential of the SNAP-tag technology according to WO2009/013359 lies within its broad range of in vitro and in vivo applications. It can be used for coupling of proteins to soluble molecules or surfaces, imaging techniques, analysis of protein-protein interaction or of pharmacokinetics in mice. Due to its versatility, a high impact of further research in the field of development of new therapeutics and diagnostics can reasonably assumed for the SNAP-tag.

The compound of the invention can be used according to the invention as a diagnostic in particular for the diagnosis of acute myeloid leukemia.

Consequently, subject matter of the present invention is also the use of the compound according to the invention in the diagnosis of acute myeloid leukemia.

Subject matter of the present invention is also a diagnostic kit comprising the polypeptide of the invention or the compound according to the invention for use in the diagnosis of acute myeloid leukemia.

### DETAILED DESCRIPTION OF THE INVENTION

As used herein, the term “antibody” refers to polyclonal antibodies, monoclonal antibodies, humanized antibodies, single-chain antibodies, and fragments thereof such as Fab, F(ab′)2, Fv, and other fragments which retain the antigen binding function and specificity of the parent antibody.

As used herein, the term “monoclonal antibody” refers to an antibody composition having a homogeneous antibody population. The term is not limited regarding the species or source of the antibody, nor is limited by the manner in which it is made. The term encompasses whole immunoglobulins as well as fragments such as Fab, F(ab′)2, Fv, and others, which retain the antigen binding function and specificity of the antibody. Monoclonal antibodies of any mammalian species can be used in this invention. In practice, however, the antibodies will typically be of rat or murine origin because of the availability of rat or murine cell lines for use in making the required hybrid cell lines or hybridomas to produce monoclonal antibodies.

As used herein, the term “human antibodies” means that the framework regions of an immunoglobulin are derived from human immunoglobulin sequences.

As used herein, the term “single chain antibody fragments” (scFv) refers to antibodies prepared by determining the binding domains (both heavy and light chains) of a binding antibody, and supplying a linking moiety, which permits preservation of the binding function. This forms, in essence, a radically abbreviated antibody, having only that part of the variable domain necessary for binding to the antigen. Determination and construction of single chain antibodies are described in U.S. Pat. No. 4,946,778 by Ladner et al.

The term “detectable label” may be any structural element which can exhibit a measurable parameter for example intrinsically by emission of radiation (radioactivity) or by interaction. Detectable labels are fluorescent dyes such as fluorescein, rhodamine, cumarine, and cyanine and derivatives hereof. Preferred fluorophores are emitting in the near infra red (NIR) range between 680 and 950 nm. This wavelength results in very low background fluorescence and excellent tissue penetration and is therefore ideally suited for fluorescence detection in vivo. In a specific embodiment a tumour specific antibody or other ligand in fusion with the Snap-tag is labeled with a O(6)-benzylguanine (BG) derivative of a NIR dye. The labeled antibody or ligand serves as an imaging tool that can be used to visualize tumor growth and/or treatment in vivo.

In a concrete example a BG derivative of an NIR dye emitting at 782 nm was coupled to a single chain antibody fragment SNAP-tag fusion protein targeting EGFR. The resulting in vivo imaging probe was used to detect EGFR expression in a pancreatic carcinoma xenograft model. In other concrete examples several fluorophore coupled complexes AB were used for flow cytometry and confocal microscopy applications. Further the detectable label can be gamma emitting radioisotopes as e.g. iodine-131, lutetium-177, yttrium 90 or any other diagnostically relevant isotope usually combined with a complexing agent as DOTA or DTAP.

Further the detectable label can be a quantum dot composed of heavy metals like CdSe or InGaP. Quantum dots are favourable optical imaging agents due to their high quantum yield and photostability. Another possibility for a fluorescent label represented by component C may be noble metal nanoclusters composed of a few (8-12) gold or silver atoms, or synthetic fluorophores captured in nanoparticles made from silicon dixode.

Further detectable labels are superparamagnetic iron oxid particles for MRI based molecular imaging.

Fluorescent proteins like GFP or dsRED or derivatives hereof can serve as detectable label coupled to the complexes AB. Fluorescent proteins today cover a wide range of the visible spectrum as well as the near infrared.

Further detectable labels can be enzymes like alkaline phosphatase, peroxidases and galactosidases that are commonly applied in a variety of immunoassays.

The terms “nonpolar amino acids”, “polar amino acids”, “neutral amino acids”, “positive amino acids” as well as “negative amino acids” designate well known properties of both essential and other amino acids. For proteinogenic amino acids the Table 1 summarises these properties:

TABLE 1

| | | | Side-chain | Side-chain charge |
|---|---|---|---|---|
| Amino Acid | 3-Letter | 1-Letter | polarity | (pH 7.4) |
| Alanine | Ala | A | nonpolar | neutral |
| Arginine | Arg | R | Basic polar | positive |
| Asparagine | Asn | N | polar | neutral |
| Aspartic acid | Asp | D | acidic polar | negative |
| Cysteine | Cys | C | nonpolar | neutral |
| Glutamic acid | Glu | E | acidic polar | negative |
| Glutamine | Gln | Q | polar | neutral |
| Glycine | Gly | G | nonpolar | neutral |
| Histidine | His | H | Basic polar | positive (10%) |
| | | | | neutral (90%) |
| Isoleucine | Ile | I | nonpolar | neutral |
| Leucine | Leu | L | nonpolar | neutral |
| Lysine | Lys | K | Basic polar | positive |
| Methionine | Met | M | nonpolar | neutral |
| Phenylalanine | Phe | F | nonpolar | neutral |
| Proline | Pro | P | nonpolar | neutral |
| Serine | Ser | S | polar | neutral |
| Threonine | Thr | T | polar | neutral |
| Tryptophan | Trp | W | nonpolar | neutral |
| Tyrosine | Tyr | Y | polar | neutral |
| Valine | Val | V | nonpolar | neutral |

Polypeptides show a peptide bond which is used to polymerise single amino acids to the biopolymer. Peptide bonds are subject to an enzymatical degradation by exo- or endopeptidases. In order to increase stability of polypeptides under natural conditions it is possible to block the N-terminal or C-terminal and/or to modify the polypeptide backbone for example by introducing peptide bonds formed by D-amino acids in particular as retro/inverso orientation.

### EXAMPLES

### Cells and Culturing

The human acute myeloid leukemia M2-derived cell line Kasumi-1 was purchased from the German Resource Centre for Biological Material (DSMZ, Braunschweig, Germany) and used as selection antigen. Cells were cultured in 80% (v/v) RPMI 1640 GlutaMAX-I medium (Invitrogen, Eggenstein, Germany) supplemented with 20% (v/v) fetal calf serum (FCS, Invitrogen) at 37° C. and 5% CO₂ and splitted every 3-4 days in a ratio of 1:2.

Beside freshly isolated human peripheral blood mononuclear cells (PBMC) from heparinised full blood using Ficoll reagent (GE Healthcare, Munchen, Germany), the human embryonic kidney cell line HEK293T and the acute myeloid leukemia M7-derived cell line KG-1 obtained from the American Type Culture Collection (ATCC, Wesel, Germany) were used as negative controls. Cells were grown in 90% (v/v) RPMI 1640 GlutaMAX-I medium containing 10% (v/v) FCS and 1% (v/v) Penicillin/Streptomycin (stock solution of 10,000 units penicillin and 10,000 μg streptomycin/ml, Invitrogen) using the same conditions as above. Additionally, HEK293T cells were used for transfection and expression of scFv-SNAP-tag fusion proteins. Therefore, cells were seeded into 24-well culture plates at a density of 6×10⁴ cells/well and incubated with 1-2 μg plasmid DNA and 3 μl FuGene HD Transfection Reagent (Roche Diagnostics GmbH, Mannheim, Germany). The expression of functional protein and the SNAP-tag activity was tested as previously described¹⁴. Successfully transfected cells were cultured under Zeocin selection pressure by adding 100 μg/ml Zeocin (InvivoGen, San Diego, Calif., USA) to the standard medium. For the production of large quantities of protein, transfected cells were cultured in triple flasks (Nunc, Langenselbold, Germany) using 200 ml medium. Medium was renewed every 7-8 days.

Soluble scFv-SNAP-tag Fusion Protein Analysis in ELISA and Flow Cytometry

The functionality of the scFv-SNAP fusion protein was demonstrated by using the crude cell culture supernatant as well as purified protein in soluble scFv ELISA. Therefore, a 96-well microtiter plate was coated overnight at 4° C. with 100 μl of a 1:100 dilution of Kasumi-1 and PBMC membrane fragments. After the plate was washed three times with PBS and blocked for 2 h with 2% MPBS, 100 μl/well of the scFv containing cell supernatant was incubated for 1 h shaking at 400 rpm at RT. Unbound protein was washed away with 0.05% PBST and bound scFv were detected via their SNAP-tag using 100 μl of freshly prepared ABTS. The substrate was added to each well and incubated in the dark as described above. The absorbance was determined at three time points (15, 30 and 60 min after the addition of ABTS) at OD_{405 nm} with reference at OD_{490 nm} in a Tecan reader.

For quantitative comparison of the binding strength of eukaryotic expressed scFv-SNAP-tag fusion proteins, 1 μg of IMAC purified protein preblocked in 2% MPBS to a total volume of 100 μl was applied in each microtiter plate well and ELISA procedure was performed as described for phage ELISA. Bound scFv-SNAP-tag fusion proteins were detected using the rabbit anti SNAP-tag polyclonal antibody (A00684, GenScript, Piscataway, N.J., USA) in a concentration of 0.2 μg/ml as primary antibody and the polyclonal goat anti rabbit HRP-labelled antibody (ab6721, Abcam, Cambridge, UK) in a dilution of 1:5000 as secondary antibody. For qualitative testing of binding activity of directly labelled scFv clones, 1 μg of the eluted scFv protein was incubated with 5×10⁵ freshly harvested and three times washed PBMCs or Kasumi-1 cells in blocking buffer (PBS containing 0.5% bovine serum albumin, BSA) for 1 h on ice protected from light. After two washing steps with PBS in a cell washer, cells were re-suspended in 300 μl blocking buffer and directly used for binding analysis in flow cytometry.

Determination of Functional Affinity Constant of the Selected scFvs

A modification of the method by Benedict et al.¹⁶ has been used for determination of affinity constants for each selected scFv antibody. The incubation of Kasumi-1 cells with various PBS-dilutions of each Vista Green labelled scFv-SNAP protein was performed as described above. Concentrations ranged from 0.5 nM-2000 nM to reach a saturation level with increasing scFv-SNAP-tag amount. After subtraction of the background fluorescent signal produced by intrinsic cell fluorescence and unspecific binding of scFv-SNAP-tag proteins, the geometric mean of the fluorescence intensity for each scFv and applied concentration was calculated. Functional affinity to PBMCs was tested in parallel to proof the specificity.

### Primary Tissue Samples

The material was archival formalin-fixed, paraffin-embedded tissue from routine histopathologic work-up. Formalin-fixation and paraffin-embedding had been performed under standardized conditions. The material had been stored with permission of the local ethics committee, after informed consent obtained from the patients prior to surgical resection. Tumor blocks of paraffin-embedded tissue were selected by two experienced gastrointestinal pathologists (Stefan Kircher, Stefan Gattenlöhner), evaluating the routine H.E. stained sections.

Immunohistochemical Staining With scFv-SNAP-tag Protein

Analysis for positive binding of selected scFv-SNAP-tag proteins was performed on serial sections of FFPE iliac crest biopsy by IHC after deparaffinization. Tissue sections were cut from formalin-fixed paraffin-embedded (FFPE) blocks on a microtome and mounted on adhesive microscope slides (Hartenstein, Wuerzburg, Germany). Staining was performed in a fully automated BOND-MAX (Leica Microsystems, Stadt, Land) using serial sections of 2 μm thickness. Slices were blocked with Peroxide Blocking reagent (Leica) for 10 min and, quickly washed three times with Bond wash solution (Leica), blocked again with 3% BSA for 20 min and washed as described before. Binding was checked by incubation with 100 μl of scFv-SNAP-tag protein containing HEK293T cell supernatant for 30 min, followed by incubation with 100 μl mouse monoclonal anti SNAP-tag antibody diluted 1:5000 in antibody diluent for 30 min. Unspecific and unbound antibodies were washed away as described above. Specific binding was visualized using Bond Polymer Refine Detection Kit according to the manufacturer's instructions. DAB staining was stopped after 10 min and cells were counterstained with hematoxylin for 5 min. After dehydration and mounting, images were taken in light microscopy. The binding signals were estimated visually by a pathologist.

### Immunofluorescence

Immunofluorescent colocalization experiments were carried out on tissue sections after deparaffinization and preparation for staining in BOND-MAX as described above. Slices were blocked with 3% BSA for 20 min, quickly washed three times with Bond washing solution (Leica) before the automated immunofluorescence pre-treatment was started. The scFv-SNAP-tag containing supernatant of transfected HEK 293T cells was cleaned from cell debris by centrifugation and mixed with monoclonal mouse anti CD34 antibody in a dilution of 1:40. After an overnight incubation on the tissue section at 4° C., unbound protein was washed away tree times with Tris buffer. Binding of scFv-SNAP-tag fusion proteins was detected via the polyclonal rabbit anti SNAP-tag antibody in a dilution of 1:1000 in Dako Diluent and subsequent incubation with goat anti rabbit Alexa Fluor 568 in a dilution of 1:500 in Tris buffer supplemented with 3% BSA. Positive binding of anti CD34 antibody was detected using the monoclonal goat unit mouse Alexa Fluor 488 in the same dilution. The incubations were performed for 2 h at room temperature with subsequent washing procedure as described. Tissue sections were mounted with Dako Fluorescence Mounting Medium and fluorescent images were taken in a microscope using the 488 nm and 568 nm filter for fluorochrome detection.

### Data Analysis

Quantitative analysis of soluble scFv proteins was carried out using AIDA image analyzer 4.27 software (Raytest, Straubenhardt, Germany) after digital scanning of Coomassie stained SDS polyacrylamide gels. Fluorophore labeled scFv were detected in VersaDoc MP System (BIO-Rad, CA, USA) and QuantityOne Basic 1-D Analysis software v4.2.1 (BIO-Rad). Data from flow cytometric analysis were evaluated using CellQuest software (Becton Dickinson, Heidelberg, Germany) and Windows Multiple Document Interface for Flow Cytometry version 2.8 (WinMDI, Joseph Trotter, USA). Statistical analysis was carried out with GraphPad Prism software (GraphPad, La Jolla, USA). Data were quoted as mean ±standard deviation (SD). A two-tailed t-test was used to determine the significance of independent experiments. The criterion p<0.05 was considered significant (*), p<0.01 very significant (**) and p<0.001 highly significant (***).

### Results

### Binding Affinity of Soluble scFv-SNAP-tag Proteins

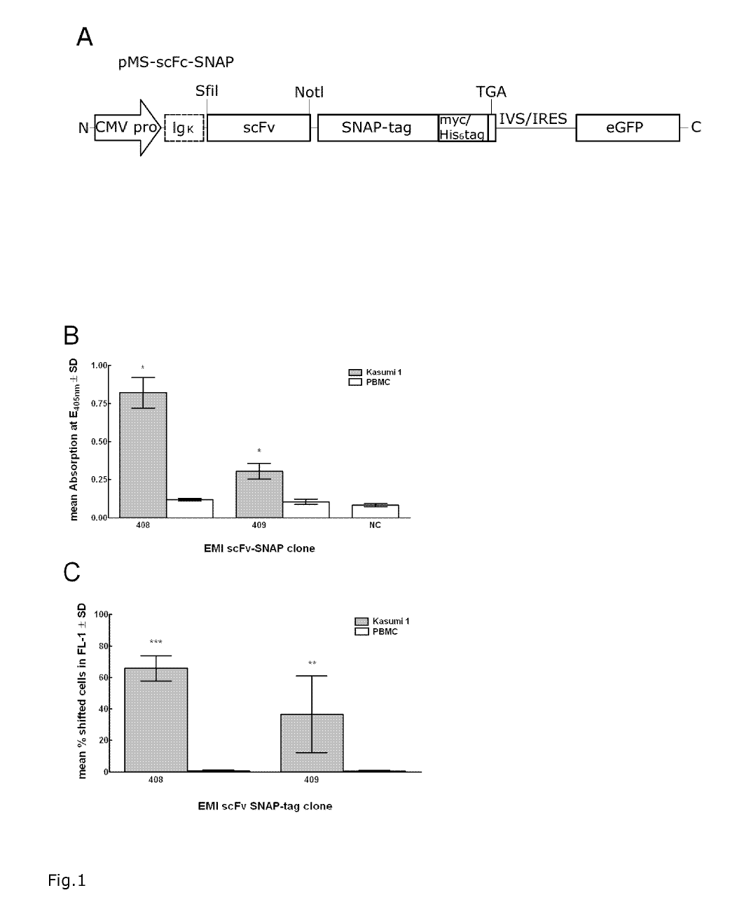

The scFv inserts were cloned into the bicistronic pMS SNAP-tag eukaryotic expression vector to generate scFv-SNAP-tag fusion proteins of the selected binders (FIG. 1A) and transfected into HEK293T cells. Effective transfection was identified by selection with Zeocin and enhanced green fluorescent (eGFP) protein activity in fluorescence microscopy. The scFv-SNAP-tag fusion proteins were secreted into the supernatant, purified via IMAC and analysed in SDS-PAGE and Western blot. The purified proteins were either used directly or after coupling to the fluorophores Vista Green or Alexa Fluor 647 using BG-SNAP substrates and labelling was successfully visualized. First classification of binding activity strength was done based on the measured absorption values in monoclonal scFv ELISA. 1 μg of each purified scFv protein was incubated with immobilized membrane fragments of Kasumi-1 and PBMC as negative control. Positive binding was detected using a rabbit anti-SNAP-tag primary antibody and a HRP-labelled goat ant rabbit secondary and visualized after the addition of ABTS at 405 nm. Selected clones with an absorption value at least 2.5 fold higher than the negative controls were classified as moderate affine, while clones with an absorption value more than 5 fold higher were declared high affine. According to this classification, the selected binder EMI408 revealed high affine binding activity to Kasumi-1 membrane fragments, clone EMI409 showed moderate binding (FIG. 1B). Additionally the binding strength to viable target cells based on flow cytometric analysis was assessed and found 36.64±24.39% for clone EMI409 and 65.75±8.07% shifted Kasumi 1 cells for clone EMI408 in FL-1 when incubated with 1 μg Vista Green labeled protein (FIG. 1C). An unspecific binding activity to PBMC depletion cells or other negative control cells like HEK293T or KG-1 was not observed at any time. The K_{D} values were determined incubating the Kasumi-1 cells with up to 2000 nM of each binder to reach a saturation level. The increasing MFIs of cell-bound scFv were measured, normalized to background fluorescence and plotted against the applied scFv concentrations in a saturation binding curve. The calculated K_{D} values of each sample using non-linear regression were 19.9±2.5 nM for clone EMI408 and to 155.8±57.3 nM for clone EMI408 (Tab. 1).

TABLE 1
Selected binders are categorized as moderate (+) or strong (++)
based on the ELISA absorption value (+ < 5×, ++ > 5× higher than
background), the percentage of shifted cells identified by FACS
(+ < 60%, ++ > 60%). Experiments were carried out at least three
times.

| | | scFv phage | | scFv SNAP | | Affinity |
|---|---|---|---|---|---|---|
| | | binder | | fusion binder | | K_{D} ± SD |
| Clone | incidence | ELISA | FACS | ELISA | FACS | in nM |
| EMI409 | 1× | + | + | + | + | 155.8 ± 57.3 |
| EMI408 | 2× | ++ | ++ | ++ | ++ | 19.9 ± 2.5 |

### Binding on FFPE Primary Tissue

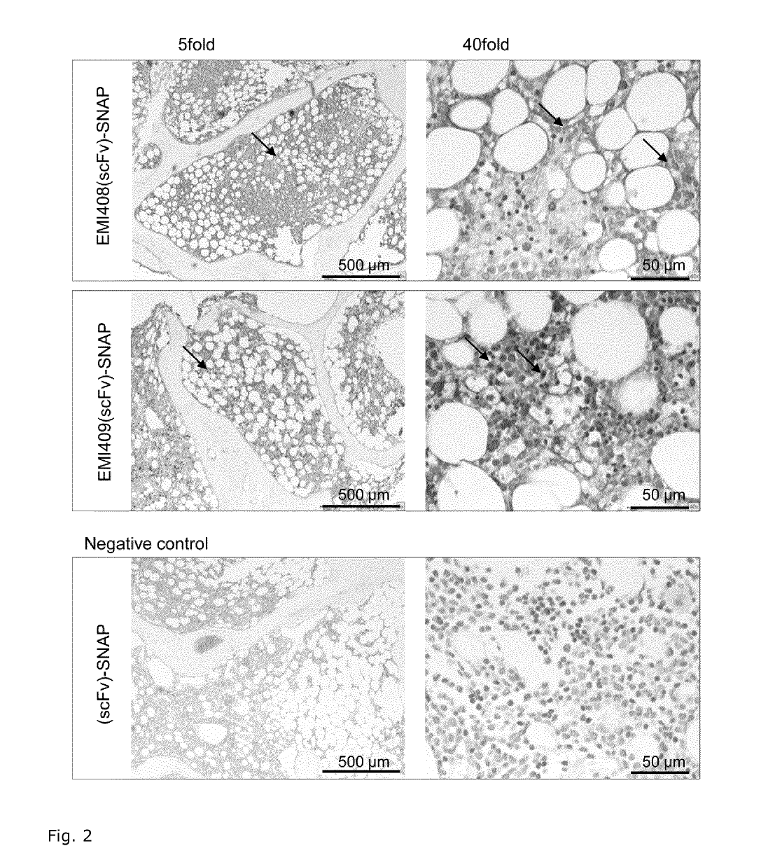

Binding analysis of the scFv-SNAP-tag containing supernatant of transfected HEK293T cells was assessed of deparaffinized FFPE tissue sections of at least 2 AML M2 positive patients. The clones EMI 408 and EMI 409 showed positive staining in IHC repeated twice. Negative control using a non-binding scFv-SNAP-tag fusion protein remained unstained neither binding on healthy bone marrow biopsy was observed (FIG. 2).

### Immunofluorescencent Double Staining

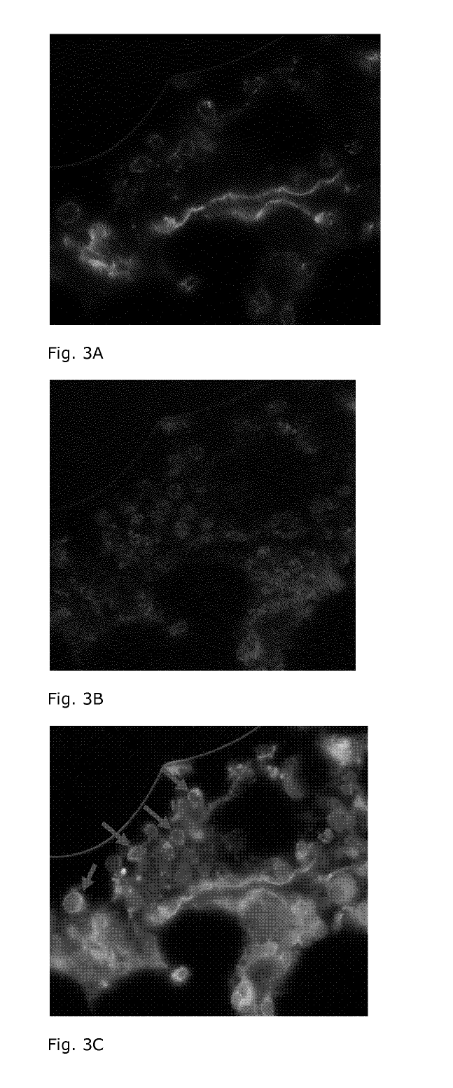

Examplary, EMI408(scFv)-SNAP-Alexa Fluor 647 was used for immunoflourescence double staining with FITC labeled anti CD34 monoclonal mouse antibody. Specific binding of clone EMI408 on the CD34 positive cell population was observed (FIG. 3).

### Cross-Reactivity of Selected scFv-SNAP-tag Fusion Proteins

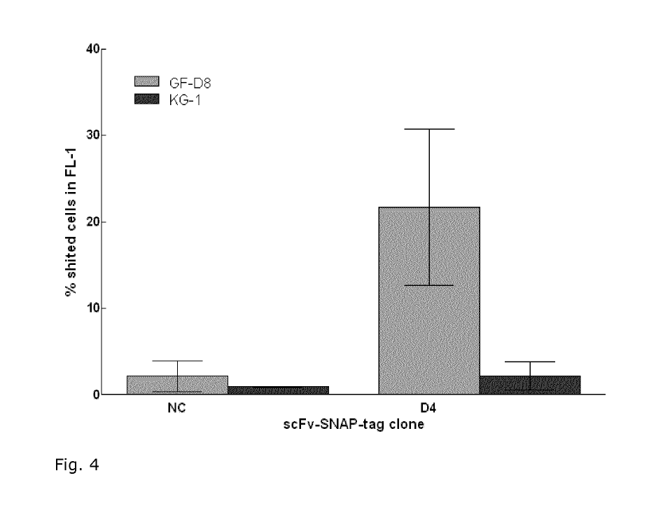

The purified and fluorophor-coupled scFv-SNAP-tag fusion proteins were checked for cross reactivity to other cell types. By use of viable flow cytometry undesired binding activity to unrelated tumor cell lines like pancreatic cancer, prostate cancer or fibroblasts could be excluded. Additionally to healthy PBMC, no binding could be observed on AML cells of other subtypes such as acute monocytic leukemia (M5, cell line: MonoMac1) and acute megakaryoblastic leukemia (M7, cell line: KG-1). However, the scFv EMI408 showed positive binding on the acute myelocytic leukemia derived cell line GF-D8 (M1) which is strongly related to the original selection cell line Kasumi-1 (M2) (FIG. 4). After incubation with 1 μg of Vista Green labeled scFv-SNAP protein EMI408 with the GF-D8 cells, 18.64±13.35% cells were shifted in FL-1. The incubation with an unspecific construct showed no signal.

### REFERENCES

(1) Abutalib, S. A., and Tallman, M. S. (2006) Monoclonal antibodies for the treatment of acute myeloid leukemia. Curr Pharm Biotechnol 7, 343-69.
(2) (2011), American Cancer Society, Atlanta.
(3) Stone, R. M. (2002) The difficult problem of acute myeloid leukemia in the older adult. CA Cancer J Clin 52, 363-71.
(4) Krebber, A., Bornhauser, S., Burmester, J., Honegger, A., Willuda, J., Bosshard, H. R., and Pluckthun, A. (1997) Reliable cloning of functional antibody variable domains from hybridomas and spleen cell repertoires employing a reengineered phage display system. J Immunol Methods 201, 35-55.
(5) Tur, M. K., Huhn, M., Jost, E., Thepen, T., Brummendorf, T. H., and Barth, S. (2011) In vivo efficacy of the recombinant anti-CD64 immunotoxin H22(scFv)-ETA′ in a human acute myeloid leukemia xenograft tumor model. Int J Cancer 129, 1277-82.
(6) Marcus, W. D., Wang, H., Lohr, D., Sierks, M. R., and Lindsay, S. M. (2006) Isolation of an scFv targeting BRG1 using phage display with characterization by AFM. Biochem Biophys Res Commun 342, 1123-9.
(7) Mulford, D. (2008) Antibody therapy for acute myeloid leukemia. Semin Hematol 45, 104-9.
(8) Pagel, J. M., Gooley, T. A., Rajendran, J., Fisher, D. R., Wilson, W. A., Sandmaier, B. M., Matthews, D. C., Deeg, H. J., Gopal, A. K., Martin, P. J., Storb, R. F., Press, O. W., and Appelbaum, F. R. (2009) Allogeneic hematopoietic cell transplantation after conditioning with 131I-anti-CD45 antibody plus fludarabine and low-dose total body irradiation for elderly patients with advanced acute myeloid leukemia or high-risk myelodysplastic syndrome. Blood 114, 5444-53.
(9) Bunjes, D., Buchmann, I., Duncker, C., Seitz, U., Kotzerke, J., Wiesneth, M., Dohr, D., Stefanic, M., Buck, A., Harsdorf, S. V., Glatting, G., Grimminger, W., Karakas, T., Munzert, G., Dohner, H., Bergmann, L., and Reske, S. N. (2001) Rhenium 188-labeled anti-CD66 (a, b, c, e) monoclonal antibody to intensify the conditioning regimen prior to stem cell transplantation for patients with high-risk acute myeloid leukemia or myelodysplastic syndrome: results of a phase I-II study. Blood 98, 565-72.
(10) ten Cate, B., Bremer, E., de Bruyn, M., Bijma, T., Samplonius, D., Schwemmlein, M., Huls, G., Fey, G., and Helfrich, W. (2009) A novel AML-selective TRAIL fusion protein that is superior to Gemtuzumab Ozogamicin in terms of in vitro selectivity, activity and stability. Leukemia 23, 1389-97.
(11) FDA. (2010), US Food and Drug Administration.
(12) Frankel, A. E., Sievers, E. L., and Scheinberg, D. A. (2000) Cell surface receptor-targeted therapy of acute myeloid leukemia: a review. Cancer Biother Radiopharm 15, 459-76.
(13) MRC. (accessed 2011).
(14) Kampmeier, F., Ribbert, M., Nachreiner, T., Dembski, S., Beaufils, F., Brecht, A., and Barth, S. (2009) Site-Specific, Covalent Labeling of Recombinant Antibody Fragments via Fusion to an Engineered Version of 6-O-Alkylguanine DNA Alkyltransferase. Bioconjug Chem 20, 1010-5.
(15) Fitting, J., Killian, D., Junghanss, C., Willenbrock, S., Murua Escobar, H., Lange, S., Nolte, I., Barth, S., and Tur, M. K. (2011) Generation of recombinant antibody fragments that target canine dendritic cells by phage display technology. Vet Comp Oncol 9, 183-95.
(16) Benedict, C. A., MacKrell, A. J., and Anderson, W. F. (1997) Determination of the binding affinity of an anti-CD34 single-chain antibody using a novel, flow cytometry based assay. J Immunol Methods 201, 223-31.
(17) Tomlinson, W. (2005) pp 82, Domantis Limited, United States.
(18) Kieke, M. C., Cho, B. K., Boder, E. T., Kranz, D. M., and Wittrup, K. D. (1997) Isolation of anti-T cell receptor scFv mutants by yeast surface display. Protein Eng 10, 1303-10.
(19) Tur, M. K., Neef, I., Jager, G., Teubner, A., Stocker, M., Melmer, G., and Barth, S. (2009) Immunokinases, a novel class of immunotherapeutics for targeted cancer therapy. Curr Pharm Des 15, 2693-9.
(20) Gao, C., Mao, S., Ronca, F., Zhuang, S., Quaranta, V., Wirsching, P., and Janda, K. D. (2003) De novo identification of tumor-specific internalizing human antibody-receptor pairs by phage-display methods. J Immunol Methods 274, 185-97.
(21) Kjaer, S., Wind, T., Ravn, P., Ostergaard, M., Clark, B. F., and Nissim, A. (2001) Generation and epitope mapping of high-affinity scFv to eukaryotic elongation factor 1A by dual application of phage display. Eur J Biochem 268, 3407-15.
(22) Guo, Z., Bi, F., Tang, Y., Zhang, J., Yuan, D., Xia, Z., and Liu, J. N. (2006) Preparation and characterization of scFv for affinity purification of reteplase. J Biochem Biophys Methods 67, 27-36.
(23) Rice, G. C., Goeddel, D. V., Cachianes, G., Woronicz, J., Chen, E. Y., Williams, S. R., and Leung, D. W. (1992) Random PCR mutagenesis screening of secreted proteins by direct expression in mammalian cells. Proc Natl Acad Sci USA 89, 5467-71.
(24) Scheuermann, R., Tam, S., Burgers, P. M., Lu, C., and Echols, H. (1983) Identification of the epsilon-subunit of Escherichia coli DNA polymerase III holoenzyme as the dnaQ gene product: a fidelity subunit for DNA replication. Proc Natl Acad Sci USA 80, 7085-9.
(25) Kortt, A. A., Dolezal, O., Power, B. E., and Hudson, P. J. (2001) Dimeric and trimeric antibodies: high avidity scFvs for cancer targeting. Biomol Eng 18, 95-108.
(26) Thompson, J., Stavrou, S., Weetall, M., Hexham, J. M., Digan, M. E., Wang, Z., Woo, J. H., Yu, Y., Mathias, A., Liu, Y. Y., Ma, S., Gordienko, I., Lake, P., and Neville, D. M., Jr. (2001) Improved binding of a bivalent single-chain immunotoxin results in increased efficacy for in vivo T-cell depletion. Protein Eng 14, 1035-41.
(27) Boehncke, W. H., Ochsendorf, F. R., Noll, S., Urban, M., Popp, A., Waldherr, D., Haunschild, J., and Litzenburger, T. (2005) Efficacy of the fully human monoclonal antibody MOR102 (#5) against intercellular adhesion molecule 1 in the psoriasis-severe combined immunodeficient mouse model. Br J Dermatol 153, 758-66.

1. A polypeptide comprising an antibody or antibody fragment for AML subtype M2 specific diagnosis wherein the polypeptide corresponds to complementarity determining regions CDR1, CDR2 and CDR3 of a heavy chain V_{H} and a light chain V_{L} of an antibody, the complementarity determining regions comprising:
the CDR 1 region of the heavy chain V_{H} defined by a sequence of 5 amino acids wherein the amino acids have side chain polarities and charges at a pH of 7.4, and the amino acids of the amino acid sequence are symbolized by a symbol as represented by a formula
PO_{N}/PO_{N}/NP_{N}/NP_{N}/PO_{N}
and the amino acids are linked via peptide bonds;
the CDR 2 region of the heavy chain V_{H} defined by a sequence of 17 amino acids wherein the amino acids have side chain polarities and charges at a pH of 7.4, and the amino acids of the amino acid sequence are symbolized by a symbol as represented by a formula
PO_{N}/NP_{N}/PO_{N}/PO_{N}/BP⁺ or NP_{N}/PO_{N}/BP⁺ or PO_{N}/BP⁺/PO_{N}/NP_{N}/PO_{N}/NP_{N}/AP⁻/PO_{N}/NP_{N}/BP⁺/PO_{N}
and the amino acids are linked via peptide bonds;
the CDR 3 region of the heavy chain V_{H} defined by a sequence of 7 amino acids wherein the amino acids have side chain polarities and charges at a pH of 7.4, and the amino acids of the amino acid sequence are symbolized by a symbol as represented by a formula
NP_{N}/NP_{N} or BP⁺/BP⁺ or NP_{N}/BP^{+ or PO}_{N}/NP_{N}/AP⁻/PO_{N}
and the amino acids are linked via peptide bonds;
the CDR 1 region of the light chain V_{L} defined by a sequence of 11 amino acids wherein the amino acids have side chain polarities and charges at a pH of 7.4, and the amino acids of the amino acid sequence are symbolized by a symbol as represented by a formula
BP⁺/NP_{N}/PO_{N}/PO_{N}/PO_{N}/NP_{N}/PO_{N}/PO_{N}/PO_{N}/NP_{N}/PO_{N}
and the amino acids are linked via peptide bonds;
the CDR 2 region of the light chain V_{L} defined by a sequence of 7 amino acids wherein the amino acids have side chain polarities and charges at a pH of 7.4, and the amino acids of the amino acid sequence are symbolized by a symbol as represented by a formula
NP_{N} or BP⁺/NP_{N}/PO_{N}/BP⁰ or NP_{N}/NP_{N}/PO_{N}/PO_{N}
and the amino acids are linked via peptide bonds; and
the CDR 3 region of the light chain V_{L} defined by a sequence of 9 amino acids wherein the amino acids have side chain polarities and charges at a pH of 7.4, and the amino acids of the amino acid sequence are symbolized by a symbol as represented by a formula
PO_{N}/PO_{N}/NP_{N} or BP⁺/BP⁺ or NP_{N}/PO_{N} or BP⁺/PO_{N}/NP_{N}/NP_{N}/PO_{N}
and the amino acids are linked via peptide bonds;
wherein the amino acids of the formulas are proteinogenic amino acids and the symbols have the meaning:
PO_{N} represents an amino acid having a polar side chain polarity and a neutral side chain charge at pH 7.4,
NP_{N} represents an amino acid having a non-polar side chain polarity and a neutral side chain charge at pH 7.4,
BP⁺ represents an amino acid having a basic polar side chain polarity and a positive side chain charge at pH 7.4,
BP⁰ represents an amino acid having a basic polar side chain polarity and a predominantly neutral side chain charge at pH 7.4, and
AP⁻ represents an amino acid having an acidic polar side chain polarity and a negative side chain charge at pH 7.4.

2. The polypeptide of claim 1, wherein:
PO_{N} represents an amino acid selected from the group consisting of asparagine, glutamine, serine, threonine, and tyrosine;
NP_{N} represents an amino acid selected from the group consisting of alanine, cysteine, glycine, isoleucine, leucine, methionine, phenylalanine, proline, tryptophane, and valine;
BP⁺ represents arginine or lysine;
BP⁰ represents histidine; and
AP⁻ represents aspartic acid or glutamic acid.

3. The polypeptide of claim 1, wherein:
the antibody or antibody fragment comprises in its heavy chain CDR 1 a peptide having at least 80% homology to the peptide of the amino acid sequence of SEQ ID NO: 1,
the antibody or antibody fragment comprises in its heavy chain CDR 2 a peptide having at least 85% homology to the peptides of the amino acid sequences SEQ ID NO: 2 or SEQ ID NO: 3, and
the antibody or antibody fragment comprises in its heavy chain CDR 3 a peptide having at least 85% homology to the peptides of the amino acid sequences SEQ ID NO: 4 or SEQ ID NO: 5.

4. The polypeptide of claim 1, wherein:
the antibody or antibody fragment comprises in its light chain CDR 1 a peptide having at least 80% homology to the peptide of the amino acid sequence of SEQ ID NO: 6,
the antibody or antibody fragment comprises in its light chain CDR 2 a peptide having at least 70% homology to the peptides of the amino acid sequences of SEQ ID NO: 7 or SEQ ID NO: 8, and
the antibody or antibody fragment comprises in its light chain CDR 3 a peptide having at least 50% homology to the peptides of the amino acid sequences of SEQ ID NO: 9 or SEQ ID NO: 10.

5. The polypeptide of claim 1, wherein:
the amino acid sequence of the heavy chain CDR 1 is the sequence of SEQ ID NO: 1,
the amino acid sequence of the heavy chain CDR 2 is the sequence of SEQ ID NO: 2 or SEQ ID NO: 3, and
the amino acid sequence of the heavy chain CDR 3 is the sequence of SEQ ID NO: 4 or SEQ ID NO: 5.

6. The polypeptide of claim 1, wherein:
the amino acid sequence of the light chain CDR 1 is the sequence of SEQ ID NO 6,
the amino acid sequence of the light chain CDR 2 is the sequence of SEQ ID NO: 7 or SEQ ID NO: 8, and
the amino acid sequence of the light chain CDR 3 is the sequence of SEQ ID NO: 9 or SEQ ID NO: 10.

7. The polypeptide of claim 1,
wherein the CDR1, CDR2 and CDR3 of the heavy chain of the variable region of an antibody v_{H} and CDR1, CDR2 and CDR3 of the light chain of the variable region of an antibody v_{L} are linked with each other via a linker structure.

8. The polypeptide of claim 1,
wherein the linker structure is (Gly4Ser)3.

9. The polypeptide of claim 1,
wherein the polypeptide is an antibody or a recombinant antibody.

10. A compound comprising the polypeptide of claim 1 comprising a detectable label.

11. The compound of claim 10,
wherein the detectable label is selected from the group consisting of fluorescent dyes, gamma ray emitting radioisotopes, a quantum dot composed of heavy metals, noble metal nanoclusters composed of at least three, super paramagnetic iron oxid particles for MRI based molecular imaging, fluorescent proteins, and enzymes.

12. The compound of claim 10,
wherein the polypeptide is linked with the detectable label by means of a chemical linking group.

13. The compound according to claim 10 for use as a diagnostic for acute myeloid leukemia subtype M2.

14. A method for diagnosing acute myeloid leukemia subtype M2 by applying the compound of claim 10 and detecting the detectable label.

15. A diagnostic kit comprising the polypeptide of claim 1 for use in the diagnosis of acute myeloid leukemia subtype M2.

16. The polypeptide of claim 9,
wherein the polypeptide is a single chain variable fragment (scFv).

17. The compound of claim 11, wherein:
the fluorescent dye is selected from the group consisting of fluorescein, rhodamine, coumarine, cyanine, and derivatives thereof;
the radioisotopes are selected from the group consisting of iodine-131, lutetium-177, and yttrium-90;
the quantum dot composed of heavy metals is selected from the group consisting of CdSe and InGaP;
the noble metal nanoclusters are selected from the group consisting of 8-12 gold or silver atoms, and synthetic fluorophores captures in nanoparticles made from silicon dioxide;
the fluorescent proteins are selected from the group consisting of GFP, dsRED, or derivatives thereof; and
the enzymes are selected from the group consisting of alkaline phosphatase, peroxidases, and galactosidases.

18. The diagnostic kit of claim 15,
wherein the polypeptide is in a compound that comprises a detectable label.


SEQUENCE LISTING (10 sequences)

SEQ ID NO: 1 - protein, 5 aa, artificial
  CDR1 of heavy chain of antibody structure
SYAMS

SEQ ID NO: 2 - protein, 17 aa, artificial
  CDR2 of heavy  chain of antibody structure
SISQRGRKTLYADSVKG

SEQ ID NO: 3 - protein, 17 aa, artificial
  CDR2 of heavy chain of antibody structure
TIGQAGSRTLYADSVKG

SEQ ID NO: 4 - protein, 7 aa, artificial
  CDR3 of heavy chain of antibody structure
GLRRFDY

SEQ ID NO: 5 - protein, 7 aa, artificial
  CDR3 of heavy  chain of antibody structure
GRATFDY

SEQ ID NO: 6 - protein, 11 aa, artificial
  CDR1 of light chain of antibody structure
RASQSISSYLN

SEQ ID NO: 7 - protein, 7 aa, artificial
  CDR2 of light chain of antibody structure of antibody structure
MASHLQS

SEQ ID NO: 8 - protein, 7 aa, artificial
  CDR2 of light chain of antibody structure
KASLLQS

SEQ ID NO: 9 - protein, 9 aa, artificial
  CDR3 of light chain of antibody structure
QQPRSTPLT

SEQ ID NO: 10 - protein, 9 aa, artificial
  CDR3 of light chain of antibody structure
QQRLRVPPT